(19) United States
(12) Patent Application Publication
(10) Pub. No.: US 2025/0248857 A1
(43) Pub. Date: Aug. 7, 2025

(54) Post-surgical garment

(71) Applicant: SILK TOUCH LASER, LLC, Boise, ID (US)

(72) Inventors: Alex Monk, Wheat Ridge, CO (US); Jason Nealy, Boise, ID (US)

(21) Appl. No.: 18/432,121

(22) Filed: Feb. 5, 2024

(51) Int. Cl. A61F 13/08; A61F 13/00; A61F 13/10; A61F 13/14; A61F 13/74

(52) U.S. Cl. CPC A61F 13/08; A61F 13/10; A61F 13/148; A61F 13/74; A61F 2013/0028

(57) ABSTRACT

A post-surgical garment includes a fabric body defining compression zone for a patient. The compression zone includes a surgical area. The fabric body includes one or more pockets that overlap at least in part with a surgical area of the patient. The one or more pockets are accessible from an outside of the garment and are configured to hold absorption pads snugly against the surgical area for absorbing fluid from the surgical area.

### BACKGROUND

The present invention relates to the field of medical garments, specifically to post-surgical garments.

Post-surgical care plays a crucial role in the recovery process of patients, influencing both healing time and patient comfort. One aspect of this care involves managing surgical incisions or wounds, which may produce fluid as part of the body's natural healing process. This fluid accumulation can lead to discomfort or potential complications if not managed properly. Traditionally, this is managed through external bandages or dressings that absorb the discharged fluids, which are changed as needed when they get saturated.

However, these dressing solutions may have several limitations. They often lack precision in targeting specific surgical areas due to their difficulty in securing to the patient's body and because of their generic shapes and sizes. As a result, they may not provide optimum absorption across different parts of the surgical site, leading to potential leakage and soiling of surrounding areas, causing discomfort and additional hygiene and infection concerns for patients.

Traditional dressings are also inconvenient for patients and medical professionals. They must be changed often, which requires disrobing, unwrapping, and then replacing and rewrapping bandages, dressings, and absorbent products. This process is messy, inconvenient, uncomfortable, and sometimes painful for the patient.

Another limitation relates to maintaining effective compression on the surgical area. Compression is known to facilitate blood circulation and reduce swelling post-surgery, plus it can help hold absorbent elements tightly against the wound area for optimal fluid management. However, traditional dressings might not always provide consistent compression across various body contours.

Garments designed specifically for post-operative use do exist in current medical practice but are generally focused on providing patient comfort and support without necessarily addressing specific needs arising from surgery-related drainage or fluid discharge effectively. Furthermore, current garments fail to account for common surgical drainage points or incision sites for various procedures (e.g., abdomen surgeries around the belly button; arm surgeries around the posterior elbow, etc.). This can lead to inefficient fluid management because absorption elements might not align correctly with these points after being placed under the garment.

Further, managing these existing post-surgical dressings through traditionally wrapped and replaced dressings is cumbersome, inconvenient, uncomfortable, and sometimes painful for the patient.

Lastly, many available solutions do not cater effectively for varied requirements based on different types of surgery procedures-a one-size-fits-all approach does not suffice when dealing with diverse surgical scenarios each having unique recovery trajectories and associated care needs.

### BRIEF SUMMARY

In some aspects, the techniques described herein relate to a post-surgical garment. The post-surgical garment includes a fabric body defining compression zone for a patient. The compression zone includes a surgical area. The fabric body includes one or more pockets that overlap at least in part with a surgical area of the patient. The one or more pockets are accessible from an outside of the garment and are configured to hold absorption pads snugly against the surgical area for absorbing fluid from the surgical area.

Various embodiments can include one or more of the following features. The one or more pockets can be configured to accommodate a foam pad to provide increased compression in the compression zone. The compression zone can includes abdomen, legs, and/or arms. The shape of each pocket can be correlated with a shape of one or more corresponding absorption pads to ensure a snug fit of the absorption pads inside the pocket.

Only the fabric body may be in direct contact with the skin of the patient at the surgical area. The pocket placement on the post-surgical garment can be correlated with common surgical drainage points. The absorption pads can be replaced without removing the garment. The pocket placement on the post-surgical garment can be correlated with common surgical incisions points including one or more of: abdomen around the belly button and lower left and lower right quadrants, arms around the posterior elbow, and legs around the medial knee and/or medial thigh.

The fabric body can be manufactured from a stretch fabric so as to create a compressive force on the compression zones, thereby supporting reduced swelling and reduced absorption pad movement. The fabric body overlapping the surgical area of the patient may be sufficiently porous to allow fluid passage through the fabric body to the absorption pad. The fabric can be made from an anti-microbial material.

The details of one or more embodiments of the invention are set forth in the accompanying drawings and the description below. Other features and advantages of the invention will be apparent from the description and drawings, and from the claims.

### BRIEF DESCRIPTION OF THE SEVERAL VIEWS OF THE DRAWINGS

In the following, the invention will be described in detail, with reference to exemplifying embodiments of the invention and to the enclosed drawings, wherein:

FIG. 1 illustrates a post-surgical garment in accordance with a first embodiment.

FIG. 2 illustrates a post-surgical garment in accordance with a second embodiment.

FIG. 3 illustrates a post-surgical garment in accordance with a third embodiment.

Like reference symbols in the various drawings indicate like elements.

### DETAILED DESCRIPTION

The various embodiments described herein relate to a post-surgical garment that offers targeted compression, fluid absorption, and potential for increased comfort to patients recovering from surgical procedures. The garment features a fabric body that outlines compression zone, which corresponds to the surgical area. Unique to this garment are pockets that overlap, at least partially, with the surgical area. These pockets, accessible from the outside of the garment, are designed to securely hold absorption pads against the surgical area, thereby facilitating the absorption of fluid from the surgical site. The garment's design ensures that only the fabric body comes into direct contact with the patient's skin at the surgical area, providing comfort alongside functionality. Having the ability to change absorption pads from the outside by merely placing a fresh absorption pad into an existing pocket greatly simplifies after-surgery care and may, in many cases, make it possible for the patient to do some of her own post-surgery care, rather than needing professional care by a nurse or similar medically trained staff to achieve proper absorption pad placement. As will be described in further detail below, the garment can have many shapes and be adapted for various areas of the body, such as the abdomen, legs, and arms, which are common surgical sites. Different embodiments of the invention can be used in various post-surgical scenarios, providing support for reduced swelling, managing fluid absorption, and reducing discomfort and potential complications for the patient. Various embodiments of the post-surgical garment will now be described by way of example and with reference to the drawings. However, it should be realized that these are merely some exemplary, and that many variations in terms of shape, size, color, fabric, pocket placement, etc., may occur.

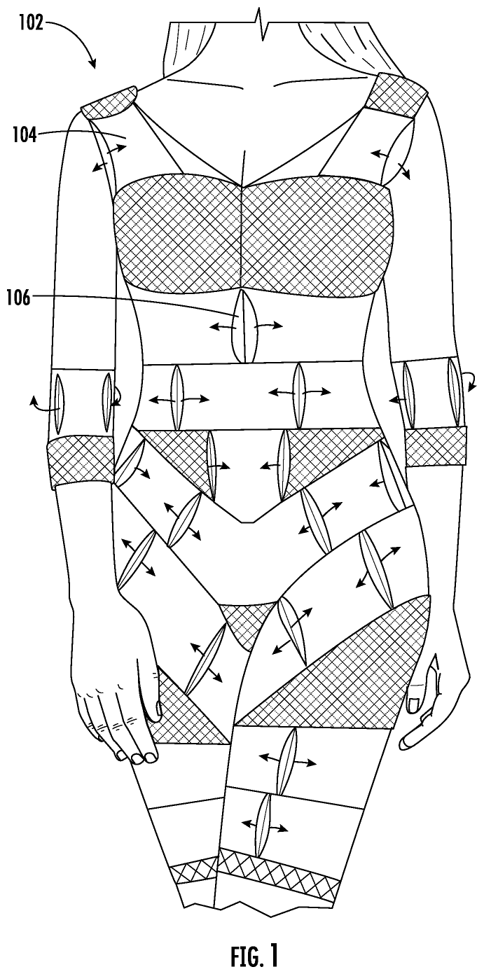

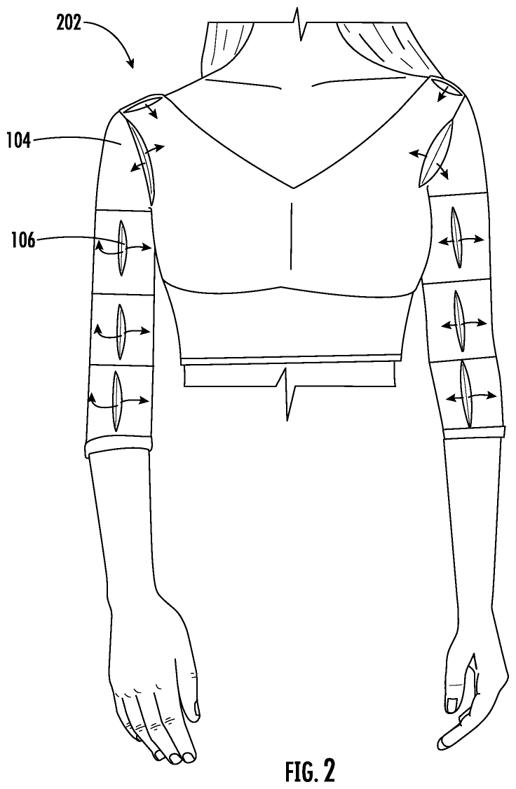

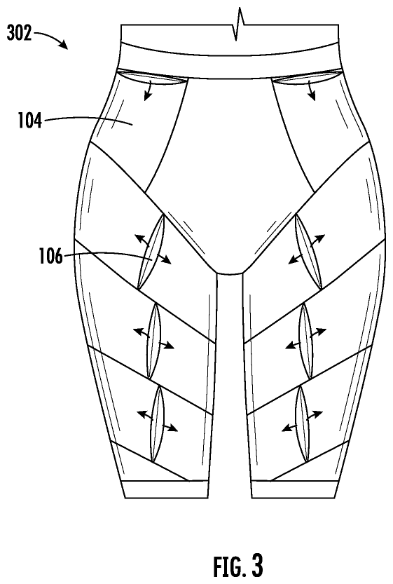

FIG. 1 illustrates a post-surgical garment 102 in accordance with a first embodiment. As shown in FIG. 1, the post-surgical garment 102 in this first embodiment covers the body from the shoulders down to the medial thighs, and medial forearms, respectively. In other embodiments, as will be described in further detail with reference to FIG. 2 and FIG. 3, respectively, the post-surgical garment may cover only the upper half or lower half of the patient's body. Even further variations may be possible that cover only a portion of an arm or leg, such as around the elbow or around the knee, for example.

The post-surgical garment 102 is manufactured from a stretch fabric and typically comes in different sizes, such as small, medium, large, extra-large, etc., to accommodate and provide an adequate amount of pressure on the patient's body. As is well known in the art, “stretch fabric” refers to a textile material that possesses the ability to stretch and recover its original shape when subjected to tension or elongation. This type of fabric is typically made using elastic fibers or by incorporating stretchable materials such as spandex, elastane, or Lycra into the fabric construction. The stretchability of the fabric allows for enhanced comfort, flexibility, and ease of movement, making it ideal for medical garments, as well as other applications such as sportswear, activewear, and undergarments.

The post-surgical garment 102 includes several pockets 104 that are designed to hold absorption pads (not shown). The absorption pads are specialized pads or materials designed specifically for medical and healthcare applications to absorb and contain bodily fluids. These pads are typically made from highly absorbent materials, such as cotton, cellulose, superabsorbent polymers, or a combination of these materials. Absorption pads are generally well-known and used in various healthcare settings like hospitals, clinics, and home care for wound management, surgical procedures, or incontinence care. They are designed to absorb bodily fluids quickly and effectively, thereby preventing leakage and maintaining a clean and dry environment. They provide an important barrier between the wound or the patient's body and the surrounding environment, helping to minimize the risk of infections and promoting proper wound healing. Absorption pads are generally available in different sizes, shapes, and absorbency levels to cater to specific needs and requirements of different medical applications. A key feature of this post-surgical garment 102 is that its pockets 104 are shaped to correlate with shapes of corresponding absorption pads. This correlation ensures a snug fit of absorption pads inside their respective pockets 104 which prevents pad displacement during movement and maximizes fluid absorption efficiency.

As shown in FIG. 1, the pockets 104 and absorption pads come in various shapes and sizes and are separated by seams in the post-surgical garment 102. The pockets 104 are placed on the post-surgical garment 102 in positions that correspond to common surgical areas, such as abdomen (in particular around the belly button and the lower left and lower right quadrants, respectively), legs (in particular around the medial knee or medial thigh), and arms (in particular around the posterior elbow). However, it should be realized that these are merely a few examples of common pocket placement, and that there may also be areas corresponding to other parts of the body where pockets may be placed. Aligning pockets with these points allows for optimal positioning of absorption pads over areas where fluid accumulation is most likely to occur after surgery. By providing targeted support and pressure on these areas following surgery, this post-surgical garment 102 facilitates improved blood circulation and minimizes inflammation, and reduces swelling in post-operative recovery.

Each pocket 104 is provided with an opening 106, schematically illustrated in FIG. 1 by arrows pointing in different directions, through which opening 106 the absorption pad can be inserted into the corresponding pocket 104. By having pockets 104 that are accessible from the outside through the openings 106, it is possible to easily change absorption pads by sliding the used absorption pads out of the pockets and sliding new absorption pads into place. This not only avoids the need for the patient to undress in order to change the wound dressing, which can be both difficult and painful following surgery, but also ensures proper placement of the absorption pads over the surgical site.

In many situations, it may even be possible for the patient herself to change the absorption pads, which may free up available time and resources in healthcare facilities, and also create increased convenience for the patient herself. Typically, the patient would be provided with written and/or verbal instructions about when and how to change absorption pads, based on the particular needs at hand, and any appropriate maintenance and/or disposal instructions for the absorption pads. In most situations, the compression provided by the post-surgical garment 102 is sufficient to keep the absorption pad in place, but in some embodiments the post-surgical garment 102 may include some kind of closure mechanism that aids in keeping the opening 106 of the pocket 104 closed after the absorption pad has been inserted.

Since the absorption pad is placed in a pocket 104, the absorption pad does not directly touch the surgical area, but instead there is a thin layer of fabric in between the incision and the absorption pad. This inside layer of fabric may be made from the same material as the rest of the post-surgical garment 102, or it may be made from a different material having different stretch properties. Irrespective, it is important that this layer, which comes in direct contact with the skin at the surgical area, exhibits sufficient porosity allowing passage of fluids and onto the absorption pad and that the porous nature facilitates efficient fluid management without compromising on comfort or fit. Since only the inside layer of fabric comes into direct contact with skin at the surgical area, this also reduces irritation and discomfort when changing absorption pads, compared to the conventional design where the absorption pads often rest directly on top of the surgical area.

In some embodiments, in addition to housing absorption pads, the pockets 104 can also accommodate additional foam pads. The inclusion of foam pads provides increased compression in the designated compression zones, when needed. This dual functionality of the pockets not only enhances fluid absorption but also aids in reducing swelling in post-operative recovery. These foam pads are typically made from medical-grade foams that are safe, hygienic, and suitable for use in clinical settings, as are well known in the art. They come in different shapes and sizes to fit the respective pockets 104 for which they are intended, and are often engineered with features like high breathability, moisture-wicking properties, and antimicrobial capabilities to promote patient comfort and reduce the risk of infections. Additionally, medical foam pads are easy to clean, maintain, and disinfect, ensuring a sterile and safe healthcare environment.

In some embodiments, the fabric used in creating the post-surgical garment 102 may be composed from an anti-microbial material offering added protection against infections during recovery period after surgery. Such fabric incorporates substances or treatments designed to inhibit the growth and spread of microorganisms such as bacteria, viruses, fungi, and mold. These fabrics are specially engineered to provide added protection against microbial contamination and potential infections. While the risks of post-surgery infections in most cases are generally low, there may be situations under which anti-microbial materials are preferred, based on the particular circumstances at hand.

FIG. 2 shows a second embodiment of a post-surgical garment 202, that is designed to be used at the upper body of a patient, in particular for the shoulder and arm regions of the patient. Just like the post-surgical garment 102 shown in FIG. 1, the post-surgical garment 202 is provided with a number of pockets 104 having openings 106 to ensure appropriate locations of absorption pads and compression zones at relevant surgical areas.

FIG. 3 shows a third embodiment of a post-surgical garment 302, that is designed to be used at the lower body of a patient, in particular for the hip and thigh regions of the patient. Just like the post-surgical garment 102 shown in FIG. 1, the post-surgical garment 302 is provided with a number of pockets 104 having openings 106 to ensure appropriate locations of absorption pads and compression zones at relevant surgical areas.

It should be noted that the post-surgical garments 202 and 302 of FIG. 2 and FIG. 3, respectively, could either be provided as separate items, or they could be parts of a “modular system” that together form a post-surgical garment similar to the post-surgical garment 102 illustrated in FIG. 1. Such a modular system might be advantageous to hospitals and other caregivers, as it may enable the same post-surgical garment 102 to be used either for a single patient or simultaneously for two different patients having had surgeries on different parts of their bodies. And again, it should be realized that the embodiments shown in FIG. 2 and FIG. 3, respectively, are merely exemplary embodiments and that there may be yet other specialized post-surgical garments for other parts of the body, such as the elbow or knee areas, for example. Thus, many variations can be envisioned by a person skilled in the art, which fall within the scope of the attached claims.

While this specification contains many details about specific embodiments, these should not be construed as limitations on the scope of the invention or of what may be claimed, but rather as descriptions of features specific to different embodiments of the invention. Certain features that are described in this specification in the context of separate embodiments can also be provided in combination in a single embodiment. Conversely, various features that are described in the context of a single embodiment can also be provided in multiple embodiments separately or in any suitable sub-combination.

Moreover, although features may be described above as acting in certain combinations and even initially claimed as such, one or more features from a claimed combination can in some cases be excised from the combination, and the claimed combination may be directed to a sub-combination or variation of a sub-combination. Thus, unless explicitly stated otherwise, or unless the knowledge of one of ordinary skill in the art clearly indicates otherwise, any of the features of one embodiment described above can be combined with any of the other features of the other embodiments described above.

Similarly, while features are depicted in the drawings in particular sizes and shapes, this should not be understood as requiring that such features be provided in the particular size and shapes, or that all illustrated features be provided, to achieve desirable results. In certain circumstances, fewer or additional features, or different sizes, shapes, and/or materials may be advantageous.

Thus, embodiments of the invention have been described. Other embodiments are within the scope of the following claims. For example, in some embodiments, the pockets may be provided with some kind of closure mechanism, such as hook and loop fasteners, buttons, flaps, zippers, etc., to keep the absorbent pad securely placed inside the pocket, and possibly to provide a more aesthetically pleasing appearances. In some embodiments, there may be labels and/or printed instructions on the pocket to aid the user in properly inserting the absorbent pads into the pockets. Thus, many variations to the above examples lie well within the scope of the attached claims and within the capabilities of a person having ordinary skill in the art.

A structure is provided between the inner (7a) and the outer tube (7b), the structure formed to provide pathway encircling the inner tube (7a) multiple times.

What is claimed is:

1. A post-surgical garment, comprising:
a fabric body defining compression zone for a patient, wherein:
the compression zone includes a surgical area,
the fabric body comprises one or more pockets that overlap at least in part with a surgical area of the patient, and
the one or more pockets are accessible from an outside of the garment and are configured to hold absorption pads snugly against the surgical area for absorbing fluid from the surgical area.

2. The post-surgical garment of claim 1, wherein the one or more pockets are further configured to accommodate a foam pad to provide increased compression in the compression zone.

3. The post-surgical garment of claim 1, wherein the compression zone includes one or more of: abdomen, legs, and arms.

4. The post-surgical garment of claim 1, wherein a shape of each pocket is correlated with a shape of one or more corresponding absorption pads to ensure a snug fit of the absorption pads inside the pocket.

5. The post-surgical garment of claim 1, wherein only the fabric body is in direct contact with the skin of the patient at the surgical area.

6. The post-surgical garment of claim 1, wherein the pocket placement on the post-surgical garment is correlated with common surgical drainage points.

7. The post-surgical garment of claim 1, wherein the absorption pads can be replaced without removing the garment.

8. The post-surgical garment of claim 1, wherein the pocket placement on the post-surgical garment is correlated with common surgical incisions points including one or more of: abdomen around the belly button and lower left and lower right quadrants, arms around the posterior elbow, and legs around the medial knee and/or medial thigh.

9. The post-surgical garment of claim 1, wherein the fabric body is manufactured from a stretch fabric so as to create a compressive force on the compression zones, thereby supporting reduced swelling and reduced absorption pad movement.

10. The post-surgical garment of claim 1, wherein the fabric body overlapping the surgical area of the patient is sufficiently porous to allow fluid passage through the fabric body to the absorption pad.

11. The post-surgical garment of claim 1, wherein the fabric is made from an anti-microbial material.
